(19) United States
(12) Patent Application Publication
(10) Pub. No.: US 2023/0277631 A1
(43) Pub. Date: Sep. 7, 2023

(54) DIAGNOSTIC METHODS FOR CANCER USING AXL DECOY RECEPTORS

(71) Applicant: Aravive Inc, Houston, TX (US)

(72) Inventors: Gail McIntyre, Lansdale, PA (US); Ray Tabibiazar, Chattanooga, TN (US); Elisabeth Gardiner, San Diego, CA (US)

(21) Appl. No.: 18/016,765

(22) PCT Filed: Jul. 18, 2021
(86) PCT No.: PCT/US21/42124

§ 371 Date: Jan. 18, 2023

Related U.S. Application Data
(60) Provisional application No. 63/053,679, filed on Jul. 19, 2020.

(51) Int. Cl. A61K 38/45; G01N 33/574; A61K 31/704; A61K 31/337; A61K 9/127; A61P 35/00; A61N 5/10

(52) U.S. Cl. CPC A61K 38/45; G01N 33/57484; A61K 31/704; A61K 31/337; A61K 9/1271; A61P 35/00; A61N 5/10; G01N 2333/912; G01N 2333/4706

(57) ABSTRACT

The present invention provides therapeutic and diagnostic methods and compositions for treating a human metastatic cancer. Specifically, the invention provides methods of treatment and methods for determining whether an individual suffering from a cancer is responding to an AXL decoy protein-based therapy, predicting responsiveness of an individual suffering from a cancer to treatment comprising an AXL decoy protein, and methods of selecting a therapy for an individual suffering from cancer.

### RELATED APPLICATIONS

This application claims benefit of U.S. Provisional Application No. 63/053,679, filed on Jul. 19, 2020, incorporated in its entirety by reference herein.

### SEQUENCE LISTING

The instant application contains a Sequence Listing in the form of a “paper copy” (PDF File) and a file containing the referenced sequences (SEQ D NOS: 1 and 2) in computer readable form (ST25 format text file) which is submitted herein. The Sequence Listing is shown using standard three letter code for amino acids, as defined in 37 C.F.R. 1.822.

### STATEMENT REGARDING FEDERALLY SPONSORED RESEARCH OR DEVELOPMENT

This work was supported by Cancer Prevention & Research Institute of Texas, New Company Product Development Award DP150127. The State of Texas, USA, may have rights in any patent issuing on this application.

### TECHNICAL FIELD

Cancer is group of diseases involving abnormal cell growth with the potential to spread or invade other parts of the body. Abnormal growths that form a discrete tumor mass, i.e., do not contain cysts or liquid areas, are defined as solid tumors. Solid tumors may be benign (not cancer), or malignant (cancer). Different types of solid tumors are named for the type of cells that form them. Examples of solid tumors are sarcomas, carcinomas, and lymphomas. Cancers derived from either of the two blood cell linages, myeloid and lymphoid, are defined as hematological malignancies. Such hematological malignancies are also referred to as blood cancers or liquid tumors. Examples of liquid tumors include multiple myeloma, acute leukemias (e.g., 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (e.g., chronic myelocytic (granulocytic) leukemia, chronic myelogenous leukemia, and chronic lymphocytic leukemia), polycythemia vera, lymphoma, Hodgkin's lymphoma, non-Hodgkin's lymphoma (indolent and high grade forms), Waldenstrom's macroglobulinemia, heavy chain disease, myelodysplastic syndrome, hairy cell leukemia and myelodysplasia.

Cancer is not a single-cell disease but rather the result of complex interactions of tumor cells with surrounding matrix and immune cells. Treatments available to cancer patients are heavily dependent on combination therapy including surgery, cytoreductive therapy and cytotoxic chemotherapy. Unfortunately, while effective in some cases, the normal tissue side effects often manifest as dose limiting toxicities and prevent tumor eradication. And even when the side effects of these various therapies can be managed, long-lasting responses are often elusive; particularly, for therapeutically refractive metastatic diseases.

In recent years, molecular targeted therapy for cancer has shown promise for many cancers. A key feature of targeted therapeutic approaches is reliable diagnostic and prognostic biomarkers. AXL has emerged as one such novel biomarker due to its role in biological processes and tumorigenesis. AXL belongs to the TAM family of receptor tyrosine kinases, which include Tyro3 (or SKY), AXL, and MER (O'Bryan, J R, Molecular and Cellular Biology, 5016-5031, 1991). The AXL receptor and its activating ligand, growth arrest-specific 6 (GAS6), are important drivers of metastasis and therapeutic resistance in human cancers. Over-expression and activation of the AXL-GAS6 signal transduction pathway has been found to be important in a wide variety of human tumors including renal, pancreatic, breast, lung, ovarian and prostate cancer (Rankin, E K, PNAS, 13373-13378, 2014) and increased expression of AXL and GAS6 in tumors has historically been correlated with poor prognosis and decreased survival and has been implicated in therapeutic resistance to conventional chemotherapeutics and targeted therapies.

Given the critical roles that GAS6 and AXL play in advanced and refractory cancers, this signaling axis represents an attractive target for therapeutic intervention. AXL-targeted drugs, either as single agents or in combination with conventional chemotherapy or other small molecule inhibitors, are likely to improve the survival of many patients. Unfortunately, an unusually strong binding affinity between GAS6 and AXL of ˜30 pM has made the development of competitive antagonists challenging. AVB-500 is a therapeutic recombinant fusion protein that has been shown to neutralize GAS6 activity by binding to GAS6 with very high affinity (see e.g., PCT WO2019/090227 wherein AVB-500 is referred to as AVB-S6-500). In doing so, AVB-500 selectively inhibits the AXL-GAS6 signaling pathway which is upregulated in multiple cancer types including ovarian cancer. In preclinical studies, AXL-GAS6 inhibition has shown anti-tumor activity in combination with a variety of anticancer therapies including radiation therapy, immuno-oncology agents, and chemotherapeutic drugs that affect DNA replication and repair. AVB-500 is currently being evaluated in clinical studies and has been granted Fast Track Designation by The U.S. Food and Drug Administration (FDA) in platinum-resistant recurrent ovarian cancer.

Patent documents Ser. Nos. 13/554,954; 13/595,936; 13/714,875; 13/950,111; 14/712,731; 14/650,852; 14/650,854; 14/910,565; 16/761,246; US2011/022125; US2013/056435; US2012/069841; US2013/074809; US2013/074786; US2013/074796; US2015/0315553 are herein specifically incorporated by reference for all teachings.

### DISCLOSURE OF THE INVENTION

The present invention is based in part on the surprising discovery that higher plasma soluble AXL/GAS6 ratios seem to correlate with response to an AXL decoy receptor (“AVB-500”) in a platinum resistant ovarian cancer study. Accordingly, the present invention provides diagnostic methods and biomarkers for pathological conditions, such as cancer, using AXL binding agents such as AVB-500.

In one aspect, the present invention provides a method of diagnosing and selecting a subject with cancer for treatment using an AXL binding agent, the method comprising: i) detecting the level of sAXL activity in a biological sample from the subject; ii) detecting the level of soluble GAS6 activity in a biological sample from the subject; and iii) selecting the subject for treatment when a sAXL/GAS6 ratio is high. In various embodiments, the sAXL/GAS6 ratio is selected from the group consisting of: greater than 0.8, greater than 0.85, greater than 0.9, greater than 0.95, greater than 1.0, greater than 1.05, greater than 1.1, greater than 1.15, greater than 1.2, greater than 1.25, greater than 1.3, greater than 1.35, greater than 1.4, greater than 1.45, greater than 1.5, greater than 2.0, greater than 2.5, and greater than 3.0. In various embodiments, the AXL binding agent is a soluble AXL variant polypeptide.

In another aspect, the present invention provides a method for treating or delaying progression of a cancer in a subject with cancer comprising administering to the subject a therapeutically effective amount of an AXL binding agent; wherein the sAXL/GAS6 ratio in a biological sample from the subject is high. In various embodiments, the sAXL/GAS6 ratio is selected from the group consisting of: greater than 0.8, greater than 0.85, greater than 0.9, greater than 0.95, greater than 1.0, greater than 1.05, greater than 1.1, greater than 1.15, greater than 1.2, greater than 1.25, greater than 1.3, greater than 1.35, greater than 1.4, greater than 1.45, greater than 1.5, greater than 2.0, greater than 2.5, and greater than 3.0. In various embodiments, the AXL binding agent is a soluble AXL variant polypeptide.

In another aspect, the present invention provides a method of diagnosing and selecting a subject with cancer for treatment using an AXL binding agent, the method comprising: i) detecting the level of sAXL phosphorylation in a biological sample from the subject; ii) detecting the level of GAS6 activity in a biological sample from the subject; and iii) selecting the subject for treatment using an AXL binding agent when the level of AXL phosphorylation and level of soluble GAS6 is high. In various embodiments, the AXL phosphorylation marker is selected from the group consisting of: Tyr698, Tyr702, Tyr703, Tyr779, Tyr821, Tyr866 and Tyr929.

In some embodiments, the AXL activity level is measured by AXL mRNA expression, the level of AXL protein expression. In some embodiments, the GAS6 activity level is measured by the level of GAS6 mRNA expression or the level of GAS6 protein expression. In some embodiments, the protein expression level is determined using a method selected from the group consisting of immunohistochemistry (IHC), immunofluorescence, flow cytometry, and Western blot. In some embodiments, the mRNA expression level is determined using a method selected from the group consisting of quantitative polymerase chain reaction (qPCR), reverse transcription qPCR (RT-qPCR), RNA sequencing, microarray analysis, in situ hybridization, and serial analysis of gene expression (SAGE).

In some embodiments, the biological sample is selected from the group consisting of a tissue sample, a blood sample, a serum sample, a plasma sample, a cerebrospinal fluid (CSF) sample, an ascites fluid sample, and a cell culture sample.

In various embodiments, the cancer is selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias. In some embodiments, the cancer is ovarian cancer. In some embodiments, the cancer is a breast cancer.

In some embodiments, the cancer is a cancer that overexpresses the biomarker GAS6 and/or AXL. In some embodiments, the cancer is a recurrent cancer. In some embodiments, the cancer is a human metastatic cancer resistant to standard therapies. In some embodiments, the human metastatic cancer is a chemoresistant cancer. In some embodiments, the human metastatic cancer is a platinum resistant cancer.

In various embodiments, the method for treating or delaying progression of a cancer in a subject further comprises a second therapy selected from the group consisting of: small molecule kinase inhibitor targeted therapy, surgery, cytoreductive therapy, cytotoxic chemotherapy, and immunotherapy. In various embodiments, the combination therapy will be synergistic. In some embodiments, the second therapy is cytoreductive therapy and the combination may increase the therapeutic index of the cytoreductive therapy. In some embodiments, the cytoreductive therapy may act in a DNA repair pathway. In some embodiments, the cytoreductive therapy is radiation therapy. In some embodiments, the combination may be synergistic.

In some embodiments, the second therapy is a chemotherapeutic agent is selected from the group consisting of: daunorubicin, adriamycin (doxorubicin), epirubicin, idarubicin, anamycin, MEN 10755, etoposide, teniposide, vinblastine, vincristine, vinorelbine (NAVELBINE); vindesine, vindoline, vincamine, mechlorethamine, cyclophosphamide, melphalan (L-sarcolysin), carmustine (BCNU), lomustine (CCNU), semustine (methyl-CCNU), streptozocin, chlorozotocin, cytarabine (CYTOSAR-U), cytosine arabinoside, fluorouracil (5-FU), floxuridine (FUdR), thioguanine (6-thioguanine), mercaptopurine (6-MP), pentostatin, fluorouracil (5-FU), methotrexate, 10-propargyl-5,8-dideazafolate (PDDF, CB3717), 5,8-dideazatetrahydrofolic acid (DDATHF), leucovorin, cisplatin (cis-DDP), carboplatin, oxaliplatin, hydroxyurea, gemcitabine, and N-methylhydrazine.

In some embodiments, the second therapy will comprise administration of a poly (ADP-ribose) polymerase inhibitor (PARP) inhibitor. In some embodiments, the PARP inhibitor is selected from the group consisting of ABT-767, AZD 2461, BGB-290, BGP 15, CEP 9722, E7016, E7449, fluzoparib, INO1001, JPI 289, MP 124, niraparib, olaparib, ONO2231, rucaparib, SC 101914, talazoparib, veliparib, WW 46, or salts or derivatives thereof. olaparib, rucaparib, niraparib, talazoparib and veliparib. In some embodiments, the combination may be synergistic.

In some embodiments, the method of treatment will comprise the administration of an AXL binding agent in combination with pegylated liposomal doxorubicin (PLD). In some embodiments, the method of treatment will comprise the administration of an AXL binding agent in combination with paclitaxel. In some embodiments, the combination may be synergistic.

In some embodiments, the second therapy will comprise immunotherapy selected from, but not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAGS, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; wherein the combination therapy provides increased effector cell killing of tumor cells, i.e., a synergy exists between the AXL binding agent and the immunotherapy when co-administered.

In some embodiments, the AXL binding agent is a soluble AXL polypeptide. In some embodiments, the soluble AXL polypeptide is a soluble AXL variant polypeptide, wherein said soluble AXL variant polypeptide lacks the AXL transmembrane domain, lacks a functional fibronectin (FN) domain, has one or more Ig1 domain, has one or more Ig2 domain, and wherein said AXL variant polypeptide exhibits increased affinity of the AXL variant polypeptide binding to GAS6 compared to wild-type AXL.

In some embodiments, the soluble AXL polypeptide is a soluble AXL variant polypeptide, wherein said soluble AXL variant polypeptide lacks the AXL transmembrane domain, lacks a functional fibronectin (FN) domain, has one Ig1 domain, lacks a functional Ig2 domain and wherein said AXL variant polypeptide exhibits increased affinity of the AXL variant polypeptide binding to GAS6 compared to wild-type AXL.

In some embodiments, the AXL variant polypeptide is a fusion protein comprising an Fc domain. In some embodiments, the variant polypeptide lacks the AXL intracellular domain. In some embodiments, the soluble AXL variant polypeptide further lacks a functional fibronectin (FN) domain and wherein said variant polypeptide exhibits increased affinity of the polypeptide binding to GAS6. In some embodiments, the soluble AXL variant polypeptide comprises at least one amino acid modification relative to the wild-type AXL sequence.

In some embodiments, the soluble AXL variant polypeptide comprises at least one amino acid modification within a region selected from the group consisting of 1) between 15-50, 2) between 60-120, and 3) between 125-135 of the wild-type AXL sequence (SEQ ID NO:1).

In some embodiments, the soluble AXL variant polypeptide comprises at least one amino acid modification at position 19, 23, 26, 27, 32, 33, 38, 44, 61, 65, 72, 74, 78, 79, 86, 87, 88, 90, 92, 97, 98, 105, 109, 112, 113, 116, 118, or 127 of the wild-type AXL sequence (SEQ ID NO: 1) or a combination thereof.

In some embodiments, the soluble AXL variant polypeptide comprises at least one amino acid modification selected from the group consisting of 1) A19T, 2) T23M, 3) E26G, 4) E27G or E27K 5) G32S, 6) N33S, 7) T38I, 8) T44A, 9) H61Y, 10) D65N, 11) A72V, 12) S74N, 13) Q78E, 14) V79M, 15) Q86R, 16) D87G, 17) D88N, 18) I90M or I90V, 19) V92A, V92G or V92D, 20) I97R, 21) T98A or T98P, 22) T105M, 23) Q109R, 24) V112A, 25) F113L, 26) H116R, 27) T118A, 28) G127R or G127E, and 29) G129E and a combination thereof.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; and (d) glycine 127.

In some embodiments, the AXL variant polypeptide comprises amino acid changes relative to the wild-type AXL sequence (SEQ ID NO: 1) at the following positions: (a) aspartic acid 87 and (b) valine 92.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; (d) glycine 127 and (e) alanine 72.

In some embodiments, the AXL variant polypeptide comprises amino acid changes relative to the wild-type AXL sequence (SEQ ID NO: 1) at the following position: alanine 72.

In some embodiments, the AXL variant polypeptide glycine 32 residue is replaced with a serine residue, aspartic acid 87 residue is replaced with a glycine residue, valine 92 residue is replaced with an alanine residue, or glycine 127 residue is replaced with an arginine residue or a combination thereof.

In some embodiments, the AXL variant polypeptide residue aspartic acid 87 residue is replaced with a glycine residue or valine 92 residue is replaced with an alanine residue or a combination thereof.

In some embodiments, the AXL variant polypeptide alanine 72 residue is replaced with a valine residue.

In some embodiments, the AXL variant polypeptide glycine 32 residue is replaced with a serine residue, aspartic acid 87 residue is replaced with a glycine residue, valine 92 residue is replaced with an alanine residue, glycine 127 residue is replaced with an arginine residue or an alanine 72 residue is replaced with a valine residue or a combination thereof.

In some embodiments, the AXL variant polypeptide comprises amino acid changes relative to the wild-type AXL sequence (SEQ ID NO: 1) at the following positions: (a) glutamic acid 26; (b) valine 79; (c) valine 92; and (d) glycine 127.

In some embodiments, the AXL variant polypeptide glutamic acid 26 residue is replaced with a glycine residue, valine 79 residue is replaced with a methionine residue, valine 92 residue is replaced with an alanine residue, or glycine 127 residue is replaced with an arginine residue or a combination thereof.

In some embodiments, the AXL variant polypeptide comprises at least an amino acid region selected from the group consisting of amino acid region 19-437, 130-437, 19-132, 21-121, 26-132, 26-121 and 1-437 of the wild-type AXL polypeptide (SEQ ID NO: 1), and wherein one or more amino acid modifications occur in said amino acid region.

In some embodiments, the AXL variant polypeptide comprises amino acid changes relative to the wild-type AXL sequence (SEQ ID NO: 1) at the following positions: (a) glycine 32; (b) aspartic acid 87; (c) alanine 72; and valine 92.

In some embodiments, the AXL variant polypeptide glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL polypeptide is a fusion protein comprising an Fc domain and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL polypeptide is a fusion protein comprising an Fc domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL variant polypeptide is a fusion protein comprising an Fc domain, lacks a functional FN domain, and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant polypeptide is a fusion protein comprising an Fc domain, lacks a functional FN domain, and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72 and (d) valine 92.

In some embodiments, the soluble AXL variant polypeptide is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, and valine 92 is replaced with an alanine residue or a combination thereof.

In some embodiments, the soluble AXL variant polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant polypeptide is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL binding agent is a fusion protein comprising using an AXL decoy receptor which comprises a soluble AXL variant polypeptide comprising amino acid changes relative to wild-type AXL sequence (SEQ ID NO:1) at the following positions: (a) glycine 32; (b) aspartic acid 87; (c) alanine 72; (d) valine 92; and (e) glycine 127, lacking the AXL transmembrane domain, lacking a functional FN domain, and comprising an Fc domain linked to the soluble AXL variant polypeptide by a peptide linker (hereinafter referred to as AVB-500).

In some embodiments, the soluble AXL variant polypeptide has an affinity of at least about 1×10⁻⁸ M, 1×10⁻⁹ M, 1×10⁻¹⁰ M, 1×10⁻¹¹ M or 1×10⁻¹² M for GAS6.

In some embodiments, the soluble AXL variant polypeptide exhibits an affinity to GAS6 that is at least about 5-fold stronger, at least about 10-fold stronger or at least about 20-fold stronger than the affinity of the wild-type AXL polypeptide.

In some embodiments, the soluble AXL variant polypeptide further comprises a linker. In some embodiments, the linker comprises one or more (GLY)₄SER units. In some embodiments, the linker comprises 1, 2, 3 or 5 (GLY)₄SER units.

In some embodiments, the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0 mg/kg, of about 19.5, and of about 20.0 mg/kg. In some embodiments, the soluble AXL variant polypeptide will be given as IV infusion over 30 or 60 minutes at a weekly dose of 20 mg/kg. In some embodiments, the soluble AXL variant polypeptide will be given as IV infusion over 30 or 60 minutes at a weekly dose of 15 mg/kg. In some embodiments, the soluble AXL variant polypeptide will be given as IV infusion over 30 or 60 minutes at a weekly dose of 10 mg/kg. In some embodiments, the soluble AXL variant polypeptide will be given as IV infusion over 30 or 60 minutes at a weekly dose of 5 mg/kg. In some embodiments, the soluble AXL variant polypeptide will be given as IV infusion over 30 or 60 minutes at a weekly dose of 2.5 mg/kg. In some embodiments, the soluble AXL variant polypeptide will be given as IV infusion over 30 or 60 minutes at a weekly dose of 1 mg/kg. In some embodiments, the soluble AXL variant polypeptide will be given as IV infusion over 30 or 60 minutes at a dose of 20 mg/kg every 14 days. In some embodiments, the soluble AXL variant polypeptide will be given as IV infusion over 30 or 60 minutes at a dose of 15 mg/kg every 14 days. In some embodiments, the soluble AXL variant polypeptide will be given as IV infusion over 30 or 60 minutes at a dose of 10 mg/kg every 14 days. In some embodiments, the soluble AXL variant polypeptide will be given as IV infusion over 30 or 60 minutes at a dose of 5 mg/kg every 14 days. In some embodiments, the soluble AXL variant polypeptide will be given as IV infusion over 30 or 60 minutes at a dose of 2.5 mg/kg every 14 days. In some embodiments, the soluble AXL variant polypeptide will be given as IV infusion over 30 or 60 minutes at a dose of 1 mg/kg every 14 days.

### BRIEF DESCRIPTION OF THE DRAWINGS

FIG. 1 is scatter plot depicting PFS as a function of sAXL/GAS6 ratio.

FIG. 2 is scatter plot depicting the correlation of baseline serum AXL/GAS6 ratio (left vertical axis) with clinical response ratio in an AVB-500 P1b platinum resistant ovarian cancer (PROC) study.

FIGS. 3A and 3B are bar graphs depicting (A) clinical response of AVB-500+PAC in patients with <3 months platinum free interval and (B) clinical response of chemotherapy in patients with <3 months platinum free interval.

FIGS. 4A and 4B are bar graphs depicting (A) clinical response of AVB-500+PAC in 3^{rd} line and 4^{th} line and (B) clinical response of chemotherapy in 3^{rd} line and 4^{th} line.

### MODE(S) FOR CARRYING OUT THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

The terms “polypeptide,” “peptide” and “protein” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms “antibody” and “antibodies” are used interchangeably herein and refer to a polypeptide capable of interacting with and/or binding to another molecule, often referred to as an antigen. Antibodies can include, for example “antigen-binding polypeptides” or “target-molecule binding polypeptides.” Antigens of the present invention can include for example any polypeptides described in the present invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. All single letters used in the present invention to represent amino acids are used according to recognized amino acid symbols routinely used in the field, e.g., A means Alanine, C means Cysteine, etc. An amino acid is represented by a single letter before and after the relevant position to reflect the change from original amino acid (before the position) to changed amino acid (after position). For example, A19T means that amino acid alanine at position 19 is changed to threonine.

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. In general, the cells of interest for detection, analysis, classification, or treatment in the present application include precancerous (e.g., benign), malignant, pre-metastatic, and non-metastatic cells.

The term “primary tumor” refers to all neoplastic cell growth and proliferation, whether malignant or benign, and all pre-cancerous and cancerous cells and tissues located at the anatomical site where the autonomous, unregulated growth of the cells initiated, for example the organ of the original cancerous tumor. Primary tumors do not include metastases.

The “pathology” of cancer includes all phenomena that compromise the well-being of the patient. This includes, without limitation, abnormal or uncontrollable cell growth, primary tumor growth and formation, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor (e.g., the organ containing the primary tumor). Metastasis can also be defined as several steps of a process, such as the departure of cancer cells from an original tumor site (e.g., primary tumor site) and migration and/or invasion of cancer cells to other parts of the body.

Depending on the nature of the cancer, an appropriate patient sample is obtained. As used herein, the phrase “cancerous tissue sample” refers to any cells obtained from a cancerous tumor. In the case of solid tumors which have not metastasized (for example a primary tumor), a tissue sample from the surgically removed tumor will typically be obtained and prepared for testing by conventional techniques.

By “early stage cancer” or “early stage tumor” is meant a cancer that is not invasive or metastatic or is classified as a Stage 0, 1, or 2 cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include bladder cancer (e.g., urothelial bladder cancer (e.g., transitional cell or urothelial carcinoma, non-muscle invasive bladder cancer, muscle-invasive bladder cancer, and metastatic bladder cancer) and non-urothelial bladder cancer), squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, as well as head and neck cancer and hematological malignancies.

Tumors of interest for treatment with the methods of the invention include solid tumors, e.g., carcinomas, gliomas, melanomas, sarcomas, and the like. Ovarian cancer and breast cancer is of particular interest. Carcinomas include a variety of adenocarcinomas, for example in prostate, lung, etc.; adrenocortical carcinoma; hepatocellular carcinoma; renal cell carcinoma, ovarian carcinoma, carcinoma in situ, ductal carcinoma, carcinoma of the breast, basal cell carcinoma; squamous cell carcinoma; transitional cell carcinoma; colon carcinoma; nasopharyngeal carcinoma; multilocular cystic renal cell carcinoma; oat cell carcinoma, large cell lung carcinoma; small cell lung carcinoma; etc. Carcinomas may be found in prostrate, pancreas, colon, brain (e.g., glioblastoma), lung, breast, skin, etc. Including in the designation of soft tissue tumors are neoplasias derived from fibroblasts, myofibroblasts, histiocytes, vascular cells/endothelial cells and nerve sheath cells. Tumors of connective tissue include sarcomas; histiocytomas; fibromas; skeletal chondrosarcoma; extraskeletal myxoid chondrosarcoma; clear cell sarcoma; fibrosarcomas, etc. Hematologic cancers include leukemias and lymphomas, e.g., cutaneous T cell lymphoma, acute myeloid leukemia (AML), chronic myeloid leukemia (CML), acute lymphoblastic leukemia (ALL), non-Hodgkins lymphoma (NHL), etc. In some embodiments, the cancer is ovarian cancer. In some embodiments, the cancer is a cancer that overexpresses the biomarker GAS6 and/or AXL. In some embodiments, the patient previously responded to treatment with an anti-cancer therapy, but, upon cessation of therapy, suffered relapse (hereinafter “a recurrent cancer”). In some embodiments, the cancer is resistant to standard therapies. In some embodiments, the cancer is a chemoresistant cancer. In some embodiments, the cancer is a platinum resistant cancer.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

The term “detection” includes any means of detecting, including direct and indirect detection.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

As used herein, when evaluating the biomarkers AXL and GAS6, an AXL/GAS6 ratio is defined as “high” when said ratio is greater than 0.8.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5 times, 2.0 times, 2.5 times, or 3.0 times the length of the treatment duration.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “objective response rate” (ORR) refers to the sum of complete response (CR) rate and partial response (PR) rate.

As used herein, “overall survival” (OS) refers to the percentage of individuals in a group who are likely to be alive after a particular duration of time.

“Inhibitors,” “activators,” and “modulators” of AXL or its ligand GAS6 (“AXL binding agents”) are used to refer to inhibitory, activating, or modulating molecules, respectively, identified using in vitro and in vivo assays for receptor or ligand binding or signaling, e.g., ligands, receptors, agonists, antagonists, and their homologs and mimetics.

The AXL binding agents having the desired pharmacological activity may be administered in a physiologically acceptable carrier to a host to modulate AXL/GAS6 function. The therapeutic agents may be administered in a variety of ways, orally, topically, parenterally e.g., intravenous, subcutaneously, intraperitoneally, by viral infection, intravascularly, etc. Intravenous delivery is of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer, including without limitation, adenocarcinoma of the ovary or prostate, breast cancer, glioblastoma, etc., including those who have undergone or are candidates for resection (surgery) to remove cancerous tissue. Subjects may be human, but also include other mammals, particularly those mammals useful as laboratory models for human disease, e.g., mouse, rat, etc.

The term “diagnosis” is used herein to refer to the identification of a molecular or pathological state, disease or condition, such as the identification of a virus infection.

As used herein, the terms “treatment,” “treating,” and the like, refer to administering an agent, or carrying out a procedure for the purposes of obtaining an effect. The effect may be prophylactic in terms of completely or partially preventing a disease or symptom thereof and/or may be therapeutic in terms of effecting a partial or complete cure for a disease, and/or symptoms of the disease. “Treatment,” as used herein, covers any treatment of any virus infection or exposure in a mammal, particularly in a human, and includes: (a) preventing the infection; (b) inhibiting the infection, i.e., arresting its development; and (c) relieving the disease, i.e., causing regression of infection.

Treating may refer to any indicia of success in the treatment or amelioration or prevention of cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions. The term “therapeutic effect” refers to the reduction, elimination, or prevention of the disease, symptoms of the disease, or side effects of the disease in the subject.

A “therapeutically effective amount” refers to the amount of a compound that, when administered to a subject for treating breast or ovarian cancer, is sufficient to affect such treatment of the cancer. The “therapeutically effective amount” may vary depending, for example, on the soluble AXL variant polypeptide selected, the stage of the cancer, the age, weight and/or health of the patient and the judgment of the prescribing physician. An appropriate amount in any given instance may be readily ascertained by those skilled in the art or capable of determination by routine experimentation.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).).

In the case of a cancer or a tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

“In combination with”, “combination therapy” and “combination products” refer, in certain embodiments, to the concurrent administration to a patient of a first therapeutic and the compounds as used herein. In some embodiments, the combination products are administered non-concurrently. When administered in combination, each component can be administered at the same time or sequentially in any order at different points in time. Thus, each component can be administered separately but sufficiently closely in time so as to provide the desired therapeutic effect.

“Concomitant administration” of a known cancer therapeutic drug with a pharmaceutical composition of the present invention means administration of the drug and AXL variant at such time that both the known drug and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

### EXEMPLARY EMBODIMENTS

In one aspect, the present invention provides a method of diagnosing and selecting a subject with cancer for treatment using an AXL binding agent, the method comprising: i) detecting the level of sAXL activity in a biological sample from the subject; ii) detecting the level of soluble GAS6 activity in a biological sample from the subject; and iii) selecting the subject for treatment when a sAXL/GAS6 ratio is high. In various embodiments, the sAXL/GAS6 ratio is selected from the group consisting of: greater than 0.8, greater than 0.85, greater than 0.9, greater than 0.95, greater than 1.0, greater than 1.05, greater than 1.1, greater than 1.15, greater than 1.2, greater than 1.25, greater than 1.3, greater than 1.35, greater than 1.4, greater than 1.45, greater than 1.5, greater than 2.0, greater than 2.5, and greater than 3.0. In various embodiments, the AXL binding agent is an AXL variant polypeptide (also referred to as “AXL decoy receptor”). In some embodiments, the soluble AXL binding agent is a fusion protein comprising using an AXL decoy receptor which comprises a soluble AXL variant polypeptide comprising amino acid changes relative to wild-type AXL sequence (SEQ ID NO:1) at the following positions: (a) glycine 32; (b) aspartic acid 87; (c) alanine 72; (d) valine 92; and (e) glycine 127, lacking the AXL transmembrane domain, lacking a functional FN domain, and comprising an Fc domain linked to the soluble AXL variant polypeptide by a peptide linker (SEQ ID NO: 2)(hereinafter referred to as AVB-500).

In another aspect, the present invention provides a method for treating or delaying progression of a cancer in a subject with cancer comprising administering to the subject a therapeutically effective amount of an AXL binding agent; wherein the sAXL/GAS6 ratio in a biological sample from the subject is high. In various embodiments, the sAXL/GAS6 ratio is selected from the group consisting of: greater than 0.8, greater than 0.85, greater than 0.9, greater than 0.95, greater than 1.0, greater than 1.05, greater than 1.1, greater than 1.15, greater than 1.2, greater than 1.25, greater than 1.3, greater than 1.35, greater than 1.4, greater than 1.45, greater than 1.5, greater than 2.0, greater than 2.5, and greater than 3.0. In various embodiments, the AXL binding agent is an AXL variant polypeptide (also referred to as “AXL decoy receptor”). In some embodiments, the soluble AXL binding agent is a fusion protein comprising using an AXL decoy receptor which comprises a soluble AXL variant polypeptide comprising amino acid changes relative to wild-type AXL sequence (SEQ ID NO:1) at the following positions: (a) glycine 32; (b) aspartic acid 87; (c) alanine 72; (d) valine 92; and (e) glycine 127, lacking the AXL transmembrane domain, lacking a functional FN domain, and comprising an Fc domain linked to the soluble AXL variant polypeptide by a peptide linker (e.g., AVB-500).

In another aspect, the present invention provides a method of diagnosing and selecting a subject with cancer for treatment using an AXL binding agent, the method comprising: i) detecting the level of sAXL phosphorylation in a biological sample from the subject; ii) detecting the level of GAS6 activity in a biological sample from the subject; and iii) selecting the subject for treatment using an AXL binding agent when the level of AXL phosphorylation and level of soluble GAS6 is high. In various embodiments, the AXL phosphorylation marker is selected from the group consisting of: Tyr698, Tyr702, Tyr703, Tyr779, Tyr821, Tyr866 and Tyr929. In various embodiments, the sAXL/GAS6 ratio is selected from the group consisting of: greater than 0.8, greater than 0.85, greater than 0.9, greater than 0.95, greater than 1.0, greater than 1.05, greater than 1.1, greater than 1.15, greater than 1.2, greater than 1.25, greater than 1.3, greater than 1.35, greater than 1.4, greater than 1.45, greater than 1.5, greater than 2.0, greater than 2.5, and greater than 3.0. In various embodiments, the AXL binding agent is an AXL variant polypeptide (also referred to as “AXL decoy receptor”). In some embodiments, the soluble AXL binding agent is a fusion protein comprising using an AXL decoy receptor which comprises a soluble AXL variant polypeptide comprising amino acid changes relative to wild-type AXL sequence (SEQ ID NO:1) at the following positions: (a) glycine 32; (b) aspartic acid 87; (c) alanine 72; (d) valine 92; and (e) glycine 127, lacking the AXL transmembrane domain, lacking a functional FN domain, and comprising an Fc domain linked to the soluble AXL variant polypeptide by a peptide linker (e.g., AVB-500).

In various embodiments, the cancer is selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

In some embodiments, the cancer is a cancer that overexpresses the biomarker GAS6 and/or AXL. In some embodiments, the cancer is a recurrent cancer. In some embodiments, the cancer is a human metastatic cancer resistant to standard therapies. In some embodiments, the human metastatic cancer is a chemoresistant cancer. In some embodiments, the human metastatic cancer is a platinum resistant cancer.

In various embodiments, the method for treating or delaying progression of a cancer in a subject further comprises a second therapy selected from the group consisting of: small molecule kinase inhibitor targeted therapy, surgery, cytoreductive therapy, cytotoxic chemotherapy, and immunotherapy. In various embodiments, the combination therapy will be synergistic. In some embodiments, the second therapy is cytoreductive therapy and the combination may increase the therapeutic index of the cytoreductive therapy. In some embodiments, the cytoreductive therapy may act in a DNA repair pathway. In some embodiments, the cytoreductive therapy is radiation therapy. In some embodiments, the combination may be synergistic.

In some embodiments, the combination therapy comprises anti-proliferative, or cytoreductive therapy. Anti-proliferative, or cytoreductive therapy is used therapeutically to eliminate tumor cells and other undesirable cells in a host and includes the use of therapies such as delivery of ionizing radiation, and administration of chemotherapeutic agents. For example, ionizing radiation (IR) is used to treat about 60% of cancer patients, by depositing energy that injures or destroys cells in the area being treated, and for the purposes of the present invention may be delivered at conventional doses and regimens, or at reduced doses. Radiation injury to cells is nonspecific, with complex effects on DNA. The efficacy of therapy depends on cellular injury to cancer cells being greater than to normal cells. Radiotherapy may be used to treat every type of cancer. Some types of radiation therapy involve photons, such as X-rays or gamma rays. Another technique for delivering radiation to cancer cells is internal radiotherapy, which places radioactive implants directly in a tumor or body cavity so that the radiation dose is concentrated in a small area. A suitable dose of ionizing radiation may range from at least about 2 Gy to not more than about 10 Gy, usually about 5 Gy. A suitable dose of ultraviolet radiation may range from at least about 5 J/m² to not more than about 50 J/m², usually about 10 J/m². The sample may be collected from at least about 4 and not more than about 72 hours following ultraviolet radiation, usually around about 4 hours.

Chemotherapeutic agents are well-known in the art and are used at conventional doses and regimens, or at reduced dosages or regimens, including for example, topoisomerase inhibitors such as anthracyclines, including the compounds daunorubicin, adriamycin (doxorubicin), epirubicin, idarubicin, anamycin, MEN 10755, and the like. Other topoisomerase inhibitors include the podophyllotoxin analogues etoposide and teniposide, and the anthracenediones, mitoxantrone and amsacrine. Other anti-proliferative agent interferes with microtubule assembly, e.g., the family of vinca alkaloids. Examples of vinca alkaloids include vinblastine, vincristine; vinorelbine (NAVELBINE); vindesine; vindoline; vincamine; etc. DNA-damaging agent include nucleotide analogs, alkylating agents, etc. Alkylating agents include nitrogen mustards, e.g., mechlorethamine, cyclophosphamide, melphalan (L-sarcolysin), etc.; and nitrosoureas, e.g., carmustine (BCNU), lomustine (CCNU), semustine (methyl-CCNU), streptozocin, chlorozotocin, etc. Nucleotide analogs include pyrimidines, e.g., cytarabine (CYTOSAR-U), cytosine arabinoside, fluorouracil (5-FU), floxuridine (FUdR), etc.; purines, e.g., thioguanine (6-thioguanine), mercaptopurine (6-MP), pentostatin, fluorouracil (5-FU) etc.; and folic acid analogs, e.g., methotrexate, 10-propargyl-5,8-dideazafolate (PDDF, CB3717), 5,8-dideazatetrahydrofolic acid (DDATHF), leucovorin, etc. Other chemotherapeutic agents of interest include metal complexes, e.g., cisplatin (cis-DDP), carboplatin, oxaliplatin, etc.; ureas, e.g., hydroxyurea; gemcitabine, and hydrazines, e.g., N-methylhydrazine. In various embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 240 mg/m², 250 mg/m², 260 mg/m², and 300 mg/m².

In some embodiments, the combination therapy will comprise immunotherapy. As used herein, the term “immunotherapy” refers to cancer treatments which include, but are not limited to treatment using depleting antibodies to specific tumor antigens (see, e.g., reviews by Blattman and Greenberg, Science, 305:200, 2004; Adams and Weiner, Nat Biotech, 23:1147, 2005; Vogal et al. J Clin Oncology, 20:719, 2002; Colombat et al., Blood, 97:101, 2001); treatment using antibody-drug conjugates (see, e.g., Ducry, Laurent (Ed.) Antibody Drug Conjugates. In: Methods in Molecular Biology. Book 1045. New York (N.Y.), Humana Press, 2013; Nature Reviews Drug Discovery 12, 259-260, April 2013); treatment using agonistic, antagonistic, or blocking antibodies to co-stimulatory or co-inhibitory molecules (immune checkpoints) such as CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, October 2014), OX-40, CD137, GITR, LAGS, TIM-3, and VISTA (see, e.g., Sharon et al., Chin J Cancer, 33(9): 434-444, September 2014; Hodi et al., N Engl J Med, 2010; Topalian et al., N Engl J Med, 366:2443-54, 2012); treatment using bispecific T cell engaging antibodies (BITE®) such as blinatumomab (see, e.g., U.S. Pat. No. 9,260,522; US Patent Application No. 20140302037); treatment involving administration of biological response modifiers such as IL-2, IL-12, IL-15, IL-21, GM-CSF, IFN-α, IFN-β, and IFN-γ□□see, e.g., Sutlu T et al., Journ of Internal Medicine, 266(2):154-181, 2009; Joshi S PNAS USA, 106(29):12097-12102, 2009; Li Y et al., Journal of Translational Medicine, 7:11, 2009); treatment using therapeutic vaccines such as sipuleucel-T (see, e.g., Kantoff P W New England Journal of Medicine, 363(5):411-422, 2010; Schlom J., Journal of the National Cancer Institutes, 104(8):599-613, 2012); treatment using dendritic cell vaccines, or tumor antigen peptide vaccines; treatment using chimeric antigen receptor (CAR)-T cells (see, e.g., Rosenberg S A Nature Reviews Cancer, 8(4):299-308, 2008; Porter D L et al, New England Journal of Medicine, 365(8):725-733, 2011; Grupp S A et al., New England Journal of Medicine, 368(16):1509-151, 2013; U.S. Pat. Nos. 9,102,761; 9,101,584); treatment using CAR-NK cells (see, e.g., Glienke et al., Front Pharmacol, 6(21):1-7, February 2015); treatment using tumor infiltrating lymphocytes (TILs)(see e.g., Wu et al, Cancer J., 18(2): 160-175, 2012); treatment using adoptively transferred anti-tumor T cells (ex vivo expanded and/or TCR transgenic)(see e.g., Wrzesinski et al., J Immunother, 33(1): 1-7, 2010); treatment using TALL-104 cells; and treatment using immunostimulatory agents such as Toll-like receptor (TLR) agonists CpG and imiquimod (see, e.g., Krieg, Oncogene, 27:161-167, 2008; Lu, Front Immunol, 5(83):1-4, March 2014).

Immunotherapy focused on utilization of depleting antibodies to specific tumor antigens have been explored with much success (see, e.g., reviews by Blattman and Greenberg, Science, 305:200, 2004; Adams and Weiner, Nat Biotech, 23:1147, 2005). A few examples of such tumor antigen-specific, depleting antibodies are HERCEPTIN® (anti-Her2/neu mAb)(Baselga et al., J Clin Oncology, Vol 14:737, 1996; Baselga et al., Cancer Research, 58:2825, 1998; Shak, Semin. Oncology, 26 (Suppl12):71, 1999; Vogal et al. J Clin Oncology, 20:719, 2002); and RITUXAN® (anti-CD20 mAb)(Colombat et al., Blood, 97:101, 2001). Unfortunately, while clearly having made a mark in oncology treatment, as monotherapy they generally work in only about 30% of the individuals and with a partial response. Moreover, many individuals eventually become refractory or relapse after treatment with these antibody-containing regimens.

Treatment using agonistic, antagonistic, or blocking antibodies to co-stimulatory or co-inhibitory molecules (immune checkpoints) has been an area of extensive research and clinical evaluation. Under normal physiological conditions, immune checkpoints are crucial for the maintenance of self-tolerance (that is, the prevention of autoimmunity) and protect tissues from damage when the immune system is responding to pathogenic infection. It is now also clear that tumors co-opt certain immune-checkpoint pathways as a major mechanism of immune resistance, particularly against T cells that are specific for tumor antigens (Pardoll D M., Nat Rev Cancer, 12:252-64, 2012). Accordingly, treatment utilizing antibodies to immune checkpoint molecules including, e.g., CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, October 2014), and OX-40, CD137, GITR, LAGS, TIM-3, and VISTA (see, e.g., Sharon et al., Chin J Cancer, 33(9): 434-444, September 2014; Hodi et al., N Engl J Med, 2010; Topalian et al., N Engl J Med, 366:2443-54) are being evaluated as new, alternative immunotherapies to treat patients with proliferative diseases such as cancer, and in particular, patients with refractory and/or recurrent cancers.

Treatment using chimeric antigen receptor (CAR) T cell therapy is an immunotherapy in which the patient's own T cells are isolated in the laboratory, redirected with a synthetic receptor to recognize a particular antigen or protein, and reinfused into the patient. CARs are synthetic molecules that minimally contain: (1) an antigen-binding region, typically derived from an antibody, (2) a transmembrane domain to anchor the CAR into the T cells, and (3) 1 or more intracellular T cell signaling domains. A CAR redirects T cell specificity to an antigen in a human leukocyte antigen (HLA)-independent fashion, and overcomes issues related to T cell tolerance (Kalos M and June C H, Immunity, 39(1):49-60, 2013). Over the last 5 years, at least 15 clinical trials of CAR-T cell therapy have been published. A new wave of excitement surrounding CAR-T cell therapy began in August 2011, when investigators from the University of Pennsylvania (Penn) published a report on 3 patients with refractory chronic lymphocytic leukemia (CLL) who had long-lasting remissions after a single dose of CAR T cells directed to CD 19 (Porter D L, et al., N Engl J Med., 365(8):725-733, 2011).

In contrast to donor T cells, natural killer (NK) cells are known to mediate anti-cancer effects without the risk of inducing graft-versus-host disease (GvHD). Accordingly, alloreactive NK cells are now also the focus of considerable interest as suitable and powerful effector cells for cellular therapy of cancer. Several human NK cell lines have been established, e.g., NK-92, HANK-1, KHYG-1, NK-YS, NKG, YT, YTS, NKL and NK3.3 (Kornbluth, J., et al., J. Immunol. 134, 728-735, 1985; Cheng, M. et al., Front. Med. 6:56, 2012) and various CAR expressing NK cells (CAR-NK) have been generated. Immunotherapy using CAR expressing NK cells (CAR-NK) is an active area of research and clinical evaluation (see, e.g., Glienke et al., Front Pharmacol, 6(21):1-7, February 2015).

Bispecific T-cell engager molecules (BiTE® s) constitute a class of bispecific single-chain antibodies for the polyclonal activation and redirection of cytotoxic T cells against pathogenic target cells. BiTE® s are bispecific for a surface target antigen on cancer cells, and for CD3 on T cells. BiTE® s are capable of connecting any kind of cytotoxic T cell to a cancer cell, independently of T-cell receptor specificity, costimulation, or peptide antigen presentation. a unique set of properties that have not yet been reported for any other kind of bispecific antibody construct, namely extraordinary potency and efficacy against target cells at low T-cell numbers without the need for T-cell co-stimulation (Baeuerle et al., Cancer Res, 69(12):4941-4, 2009). BiTE antibodies have so far been constructed to more than 10 different target antigens, including CD19, EpCAM, Her2/neu, EGFR, CD66e (or CEA, CEACAM5), CD33, EphA2, and MCSP (or HMW-MAA)(Id.) Treatment using BiTE® antibodies such as blinatumomab (Nagorsen, D. et al., Leukemia & Lymphoma 50(6): 886-891, 2009) and solitomab (Amann et al., Journal of Immunotherapy 32(5): 452-464, 2009) are being clinically evaluated.

In some embodiments, the second therapy will comprise administration of a PARP inhibitor. Poly(ADP-ribose) polymerases (PARPs) are a family of enzymes involved in various activities in response to DNA damage. PARP-1 is a key DNA repair enzyme that mediates single strand break (SSB) repair through the base excision repair (BER) pathway. PARP inhibitors have been demonstrated to selectively kill tumor cells that harbor BRCA1 and BRCA2 mutations. In addition, pre-clinical and preliminary clinical data suggest that PARP inhibitors are selectively cytotoxic for tumors with homologous recombination repair deficiency caused by dysfunction of genes other than BRCA1 or BRCA2. In some embodiments, the PARP inhibitor is selected from the group consisting of ABT-767, AZD 2461, BGB-290, BGP 15, CEP 9722, E7016, E7449, fluzoparib, INO1001, JPI 289, MP 124, niraparib, olaparib, ONO2231, rucaparib, SC 101914, talazoparib, veliparib, WW 46, or salts or derivatives thereof. In some embodiments, the anti-PARP therapy is administered at a dose equivalent to about 100 mg, about 200 mg, or about 300 mg of niraparib or a salt or derivative thereof. In some embodiments, the anti-PARP therapy is administered at a dose equivalent to about 100 mg of niraparib or a salt or derivative thereof. In some embodiments, the anti-PARP therapy is administered at a dose equivalent to about 200 mg of niraparib or a salt or derivative thereof. In certain embodiments, the anti-PARP therapy is administered at a dose equivalent to about 300 mg of niraparib or a salt or derivative thereof.

The AXL variant may be administered prior to, concurrently with, or following the second therapy, usually within at least about 1 week, at least about 5 days, at least about 3 days, at least about 1 day. The AXL variant may be delivered in a single dose, or may be fractionated into multiple doses, e.g., delivered over a period of time, including daily, bidaily, semi-weekly, weekly, etc. The effective dose will vary with the route of administration, the specific agent, the dose of cytoreductive agent, and the like, and may be determined empirically by one of skill in the art. A useful range for i.v. administered polypeptides may be empirically determined, for example at least about 0.1 mg/kg body weight; at least about 0.5 mg/kg body weight; at least about 1 mg/kg body weight; at least about 2.5 mg/kg body weight; at least about 5 mg/kg body weight; at least about 10 mg/kg body weight; at least about 20 mg/kg body weight; or more. In some embodiments, the soluble AXL variant polypeptide will be given as IV infusion over 30 or 60 minutes at a weekly dose of 20 mg/kg. In some embodiments, the soluble AXL variant polypeptide will be given as IV infusion over 30 or 60 minutes at a weekly dose of 15 mg/kg. In some embodiments, the soluble AXL variant polypeptide will be given as IV infusion over 30 or 60 minutes at a weekly dose of 10 mg/kg. In some embodiments, the soluble AXL variant polypeptide will be given as IV infusion over 30 or 60 minutes at a weekly dose of 5 mg/kg. In some embodiments, the soluble AXL variant polypeptide will be given as IV infusion over 30 or 60 minutes at a weekly dose of 2.5 mg/kg. In some embodiments, the soluble AXL variant polypeptide will be given as IV infusion over 30 or 60 minutes at a weekly dose of 1 mg/kg. In some embodiments, the soluble AXL variant polypeptide will be given as IV infusion over 30 or 60 minutes at a dose of 20 mg/kg every 14 days. In some embodiments, the soluble AXL variant polypeptide will be given as IV infusion over 30 or 60 minutes at a dose of 15 mg/kg every 14 days. In some embodiments, the soluble AXL variant polypeptide will be given as IV infusion over 30 or 60 minutes at a dose of 10 mg/kg every 14 days. In some embodiments, the soluble AXL variant polypeptide will be given as IV infusion over 30 or 60 minutes at a dose of 5 mg/kg every 14 days. In some embodiments, the soluble AXL variant polypeptide will be given as IV infusion over 30 or 60 minutes at a dose of 2.5 mg/kg every 14 days. In some embodiments, the soluble AXL variant polypeptide will be given as IV infusion over 30 or 60 minutes at a dose of 1 mg/kg every 14 days.

In some embodiments, the treatment regime entails administration once per every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In still some embodiments, therapeutic entities of the present invention are often administered as pharmaceutical compositions comprising an active therapeutic agent, i.e., and a variety of other pharmaceutically acceptable components. (See Remington's Pharmaceutical Science, 15.sup.th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions of the present invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still yet some other embodiments, for prophylactic applications, pharmaceutical compositions or medicaments are administered to a patient susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

In still yet some other embodiments, for therapeutic applications, therapeutic entities of the present invention are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically effective dose. In both prophylactic and therapeutic regimes, agents are usually administered in several dosages until a sufficient response has been achieved. Typically, the response is monitored, and repeated dosages are given if there is a recurrence of the cancer.

According to the present invention, compositions for the treatment of primary or metastatic cancer can be administered by parenteral, topical, intravenous, intratumoral, oral, subcutaneous, intraarterial, intracranial, intraperitoneal, intranasal, or intramuscular means. The most typical route of administration is intravenous or intratumoral although other routes can be equally effective.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. In some embodiments, the composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein. Alternatively, transdermal delivery can be achieved using a skin patch or using transfersomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration. Preferably, a therapeutically effective dose of the polypeptide compositions described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Also within the scope of the invention are kits comprising the compositions of the invention and instructions for use. The kit can further contain a least one additional reagent, for example a cytoreductive drug. The compositions may be provided in a unit dose formulation. Kits typically include a label indicating the intended use of the contents of the kit. The term label includes any writing, or recorded material supplied on or with the kit, or which otherwise accompanies the kit.

### Example 1

The present inventors have recently completed a Phase 1b dose-escalation study (AVB500-OC-002) evaluating AVB-500 in combination with pegylated liposomal doxorubicin (PLD) or Paclitaxel (Paclitaxel) for patients with platinum resistant ovarian cancer (PROC). The dosing protocol was AVB-500 given as IV infusion over 60 minutes, starting on Day 1, for a 28-day treatment cycle. Physician's choice of chemotherapy can follow but currently included 1) Paclitaxel given weekly as IV infusion over 60 minutes at a dose of 80 mg/m² for a 28-day treatment cycle or 2) PLD given as IV infusion over 60 minutes at a dose of 40 mg/m² on Day 1 of a 28-day treatment cycle. Patient selection includes ovarian cancer patients with 1-3 prior lines of therapy, advanced, measurable disease, with a platinum free interval ≤6 mo after most recent platinum-containing regimen. Positive data from the Phase 1b study was shown at 10 mg/kg, and a relationship was demonstrated showing a strong correlation between AVB-500 blood levels and anti-tumor response in the platinum resistant patients.

The safety of AVB-500 has been studied in 84 subjects, including 31 healthy volunteers in a Phase 1a study and 53 patients with PROC in a Phase 1 b study (40 in 10 mg/kg cohort, 6 in 15 mg/kg cohort, and 7 in 20 mg/kg cohort). The primary objectives of the PROC study were to assess safety and pharmacokinetics of AVB-500 in combination with paclitaxel (PAC) or pegylated liposomal doxorubicin (PLD). Secondary endpoints include objective response rate (ORR), CA-125 response, disease control rate, progression free survival (PFS), overall survival, pharmacokinetic (PK) profile, GAS6 serum levels, and anti-drug antibody titers. Analysis of all safety data to date demonstrates that AVB-500 is well-tolerated with no dose-limiting toxicities or unexpected safety signals. There have been no AVB-500-related SAEs reported to date.

Prior data analysis of 31 patients from the 10 mg/kg cohort showed that blood trough levels of AVB-500 demonstrated statistically significant correlation with efficacy as patients who achieved minimal efficacious concentration (MEC)>13.8 mg/L demonstrated a greater likelihood of response and prolonged PFS. Updated modeling using actual data from all enrolled patients demonstrated that 60%, 85%, and 93% of patients achieved MEC at doses of 10 mg/kg, 15 mg/kg, and 20 mg/kg, respectively.

Preliminary efficacy can be summarized as follows: in the 10 mg/kg cohort (37 out of 40 patients evaluable): 31% ORR (5/16) among those treated with AVB-500 in combination with PAC, with 1 complete response (CR). Patients given AVB-500 plus PAC who achieved MEC of AVB-500 demonstrated improved ORR of 50% (4/8), with 1 CR. The PFS among those who achieved MEC of AVB-500 was 7.5 months versus 2.75 months with those below MEC (p=0.0062). 21.6% ORR (8/37) in all evaluable patients, regardless of their MEC or use of PAC or PLD. In the 15 mg/kg cohort (5 out of 6 patients evaluable): all 5 patients in this cohort demonstrated clinical benefit, with 1 CR (continuing to show CR 3 months after discontinuing chemotherapy while on AVB-500 as single agent), 2 PR, and 2 SD. In the 20 mg/kg cohort (7 out of 7 patients evaluable): Of the 7 patients in this cohort, there was 1 deep PR (with CR of target lesion; not confirmed), 1 SD, and 5 with PD. 15 mg/kg has been chosen for use as the recommended phase 2 dose.

Table 1 shows the overall patient responses to the targeted therapeutic AVB-500 in combination with Paclitaxel (PAC) or PLD.

TABLE 1
Patient Responses to AVB-500 + Paclitaxel
(PAC) vs AVB-500 + PLD

| Patient Response | AVB-500 + PAC | AVB-500 + PLD |
|---|---|---|
| Complete Response (CR) | 14% | 0% |
| Partial Response (PR) | 28% | 12% |
| Stable Disease (SD) | 21% | 65% |
| Progressive Disease (PD) | 36% | 24% |
| Overall Response Rate (ORR) | 35% | 15% |

Notably, it appears that AVB-500 plus PAC performs better than AVB-500 plus PLD, as AVB-500 plus PAC data show an ORR of 35% (8/23, including 2 CRs) compared to ORR of 15% (4/26) in AVB-500 plus PLD in patients. It also appears that AVB-500 plus chemo appears to perform better in patients without previous exposure to bevacizumab, as in a subgroup analysis of patients who had not been previously exposed to bevacizumab in their prior lines of therapy, AVB-500 yields an ORR of 60% (6/10 including 2 CR) when combined with PAC and an ORR of 19% (3/16) when combined with PLD. For reference, control arms of the AURELIA Study (NCT00976911) showed ORR of 30.2% (16/55) with PAC alone and 7.8% (5/64) with PLD alone.

To evaluate a biomarker strategy, patient tissue was examined from the time of patient diagnosis. This approach addressed the presence of soluble AXL in tissue at the time of initial debulking surgery or clinical biopsy. This was done in conjunction with the use of a proprietary pharmacodynamic (PD) assay for serum GAS6 that was developed to guide dose selection in clinical trials for AVB-500. The two assays together can be used to establish the specific criteria for treatment and patient selection for this targeted therapy. This assay also appears to have utility in monitoring therapeutic response, allowing clinicians to be nimble and guided for better treatment timing and opening the progression free survival (PFS) and over response rate (ORR) for patients.

Table 2 shows the overall patient responses to the targeted therapeutic AVB-500 in combination with Paclitaxel as relates to baseline serum sAXL/GAS6 ratio.

TABLE 2
sAXL/GAS6 RATIO AVB-500 + PAC (10 & 15 mg/kg Patients)

| Ratio | Above 0.773 | Below 0.773 |
|---|---|---|
| N | 12 | 7 |
| CR | 2 (17%) | 0 |
| PR | 5 (42%) | 0 |
| ORR | 7 (58%) | 0 |
| SD | 2 (17%) | 4 (57%) |
| CBR | 9 (75%) | 4 (57%) |
| PD | 3 (25%) | 3 (43%) |
| mPFS | 6.6 | 2.9 |
| mOS | 19.0 | 9.2 |

CBR = clinical benefit rate (ORR + SD);
mOS = median overall survival;
mPFS = median progressive free survival

Table 3 shows the overall patient responses to the targeted therapeutic AVB-500 in combination with Paclitaxel as relates to baseline serum sAXL/GAS6 ratio including if bevacizumab naïve or previously received bevacizumab.

TABLE 3
Efficacy Data for patients receiving 10 & 15 mg/kg
AVB-500 + PAC including if bevacizumab
naïve or previously received bevacizumab

| | >MEC** | <MEC** | Bev Naïve | Previous Bev |
|---|---|---|---|---|
| | (N = 10) | (N = 9) | (N = 9) | (N = 10) |
| ORR | 5 (50%), | 2 (22%) | 6 (67%), | 1 (10%) |
| | 2 CRs\hat{ } | | 2 CRs\hat{ } | |
| mPFS (months) | 7.5 | 2.8 | 7.7 | 2.8 |
| mOS (months) | 19 | 8.7 | 19.3 | 9.2 |

* Study ongoing so data can change;
**MEC = minimal efficacious concentration (13.8 mg/L)
\hat{ }1 patient at 10 mg/kg and 1 patient at 15 mg/kg had CR; 1 patient at 15 mg/kg with CR continues to show CR at C13D1 while on AVB-500 alone, 6 months after discontinuing paclitaxel

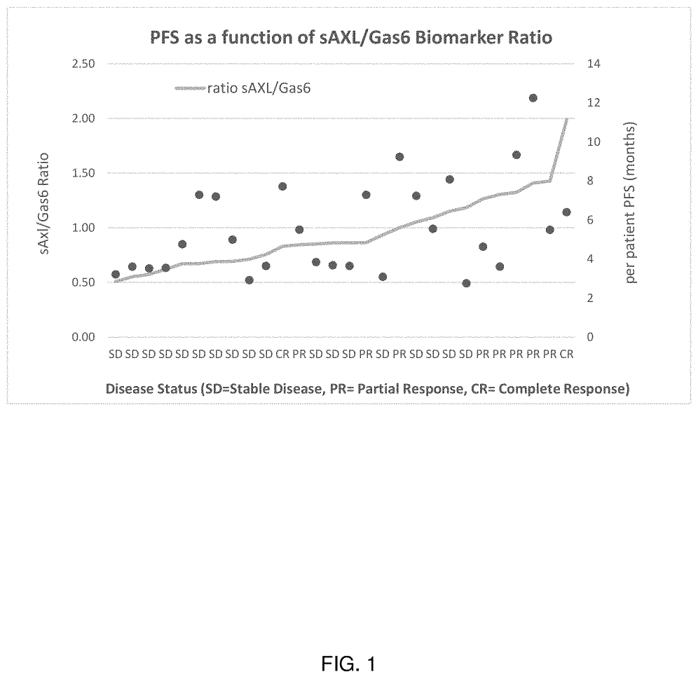

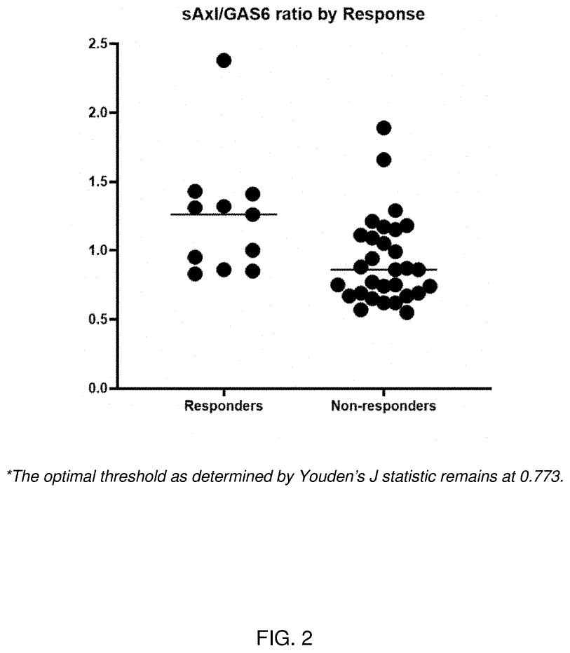

When examining response by dose and looking at the ratio of sAXL/GAS6 in patients as a function of response, a clear pattern of the utility of this biomarker strategy emerged in the study. Plasma levels of sAXL/GAS6 ratio seem to correlate well with response to AVB-500 (FIG. 1) and this ratio metric approach has emerged as a key tool for future patient stratification. As depicted in FIG. 2 and Table 2, serum sAXL/GAS6 ratio correlates well with response to AVB-500+chemo and remains a significant predictor of RECIST response in this sample (AUC=0.7833; p=0.047). Patients with A/G ratios above 0.773 were significantly more likely to have achieved a RECIST response with a calculated sensitivity of 100% and a specificity of 60%.

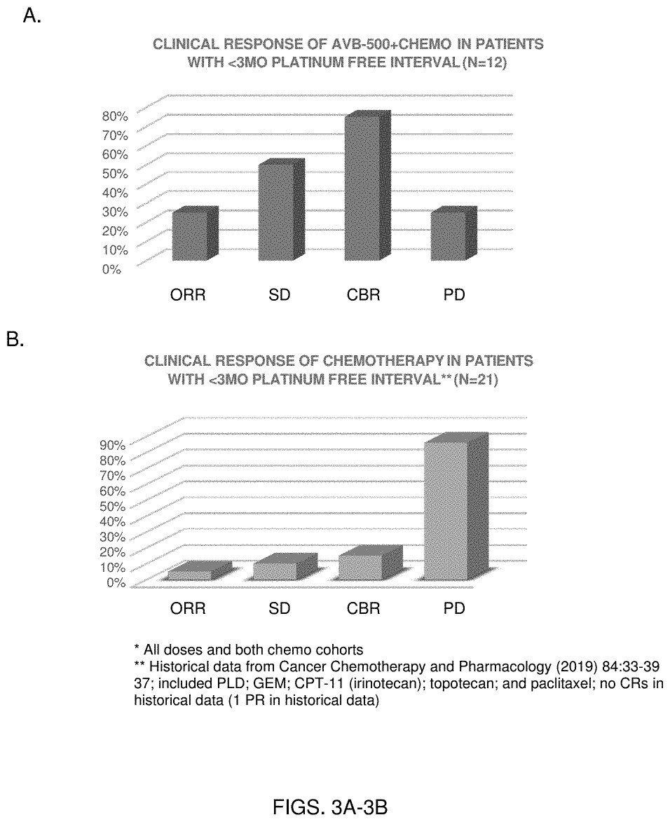

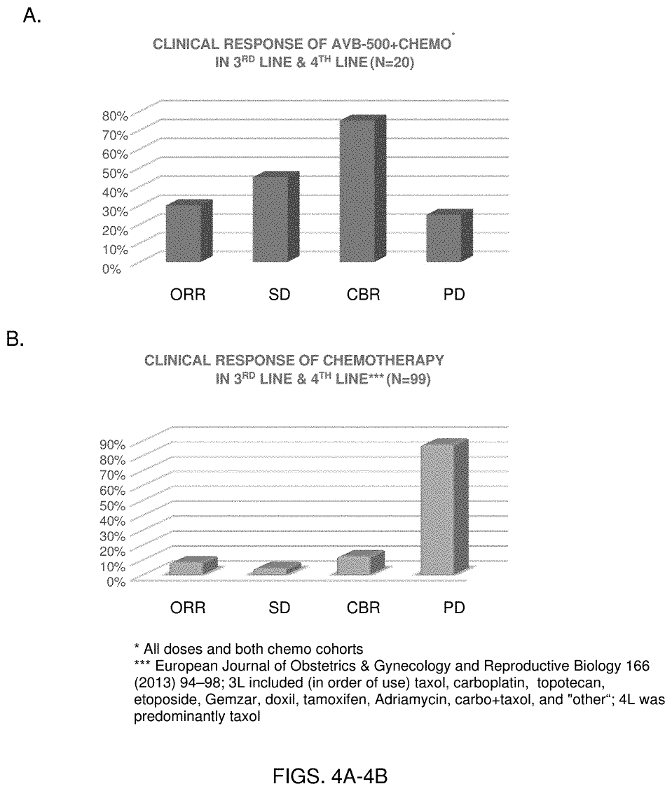

In the entire Phase 1 b cohort, patients with a high sAXL/GAS6 ratio had 30% ORR (10/33) versus 0% ORR (0/15) in low AXL/GAS6. In the PAC cohort, patients with a high sAXL/GAS6 ratio had 43% ORR (6/14) versus 0% ORR (0/7) with low sAXL/GAS6. Notably, patients with high sAXL/GAS6 ratio who had not previously received bevacizumab achieved ORR of 75% (6/8). Historically, high sAXL is associated with a poor prognosis. Surprisingly, however, AVB-500 plus PAC or PLD appears to improve clinical outcomes in this population (see FIGS. 3 and 4).

By extension, this data provides relevance to tissue or serum AXL and GAS6 present in serum or tissue as a prognostic or diagnostic marker of disease pathology and patient receptivity to targeted treatment by AXL decoy proteins. And importantly, despite the historical association of high AXL expression with poor cancer prognosis, AVB-500 appears to have more activity in the elevated sAXL population and suggests that using a sAXL/GAS6 ratio as a biomarker approach will provide better patient stratification and will be a prognostic marker that will uniquely inform clinicians and provide focused treatment to cancer patients using this targeted pharmaceutical agent, thus advancing the state of the art.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize or be able to ascertain using no more than routine experimentation, many equivalents to the specific embodiments of the invention described herein. Such equivalents are intended to be encompassed by the appended claims.

### Sequence Listings

The nucleic and amino acid sequences listed in the accompanying sequence listing are shown using standard letter abbreviations for nucleotide bases and three letter code for amino acids, as defined in 37 C.F.R. 1.822.

Human AXL polypeptide amino acid sequence
SEQ ID NO: 1
MGRVPLAWCLALCGWACMAPRGTQAEESPFVGNPGNITGARGLTGTLRCQL
QVQGEPPEVHWLRDGQILELADSTQTQVPLGEDEQDDWIVVSQLRITSLQL
SDTGQYQCLVFLGHQTFVSQPGYVGLEGLPYFLEEPEDRTVAANTPFNLSC
QAQGPPEPVDLLWLQDAVPLATAPGHGPQRSLHVPGLNKTSSFSCEAHNAK
GVTTSRTATITVLPQQPRNLHLVSRQPTELEVAWTPGLSGIYPLTHCTLQA
VLSNDGMGIQAGEPDPPEEPLTSQASVPPHQLRLGSLHPHTPYHIRVACTS
SQGPSSWTHWLPVETPEGVPLGPPENISATRNGSQAFVHWQEPRAPLQGTL
LGYRLAYQGQDTPEVLMDIGLRQEVTLELQGDGSVSNLTVCVAAYTAAGDG
PWSLPVPLEAWRPGQAQPVHQLVKEPSTPAFSWPWWYVLLGAVVAAACVLI
LALFLVHRRKKETRYGEVFEPTVERGELVVRYRVRKSYSRRTTEATLNSLG
ISEELKEKLRDVMVDRHKVALGKTLGEGEFGAVMEGQLNQDDSILKVAVKT
MKIAICTRSELEDFLSEAVCMKEFDHPNVMRLIGVCFQGSERESFPAPVVI
LPFMKHGDLHSFLLYSRLGDQPVYLPTQMLVKFMADIASGMEYLSTKRFIH
RDLAARNCMLNENMSVCVADFGLSKKIYNGDYYRQGRIAKMPVKWIAIESL
ADRVYTSKSDVWSFGVTMWEIATRGQTPYPGVENSEIYDYLRQGNRLKQPA
DCLDGLYALMSRCWELNPQDRPSFTELREDLENTLKALPPAQEPDEILYVN
MDEGGGYPEPPGAAGGADPPTQPDPKDSCSCLTAAEVHPAGRYVLCPSTTP
SPAQPADRGSPAAPGQEDGA
Exemplary soluble AXL polypeptide-Fc fusion.
SEQ ID NO: 2
EESPFVSNPGNITGARGLTGTLRCQLQVQGEPPEVHWLRDGQILELVDSTQ
TQVPLGEDEQGDWIVASQLRITSLQLSDTGQYQCLVFLGHQTFVSQPGYVR
LEGLPYFLEEPEDRTVAANTPFNLSCQAQGPPEPVDLLWLQDAVPLATAPG
HGPQRSLHVPGLNKTSSFSCEAHNAKGVTTSRTATITVLPQQGGGGSDKTH
TCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVM
HEALHNHYTQKSLSLSPG

1-26. (canceled)

27. A method of diagnosing and selecting a subject with cancer for treatment using an AXL binding agent, the method comprising: i) detecting the level of sAXL activity in a biological sample from the subject; ii) detecting the level of soluble GAS6 activity in a biological sample from the subject; and iii) selecting the subject for treatment when a sAXL/GAS6 ratio is high; wherein the sAXL/GAS6 ratio is selected from the group consisting of: greater than 0.8, greater than 0.85, greater than 0.9, greater than 0.95, greater than 1.0, greater than 1.05, greater than 1.1, greater than 1.15, greater than 1.2, greater than 1.25, greater than 1.3, greater than 1.35, greater than 1.4, greater than 1.45, greater than 1.5, greater than 2.0, greater than 2.5, and greater than 3.0.

28. A method of diagnosing and selecting a subject with cancer for treatment using an AXL binding agent, the method comprising: i) detecting the level of sAXL phosphorylation in a biological sample from the subject; ii) detecting the level of GAS6 activity in a biological sample from the subject; and iii) selecting the subject for treatment using an AXL binding agent when the level of AXL phosphorylation and level of soluble GAS6 is high; wherein the AXL phosphorylation marker is selected from the group consisting of: Tyr698, Tyr702, Tyr703, Tyr779, Tyr821, Tyr866 and Tyr929.

29. A method for treating or delaying progression of a cancer in a subject with cancer comprising administering to the subject a therapeutically effective amount of an AXL binding agent; wherein the sAXL/GAS6 ratio in a biological sample from the subject is high; wherein the sAXL/GAS6 ratio is selected from the group consisting of: greater than 0.8, greater than 0.85, greater than 0.9, greater than 0.95, greater than 1.0, greater than 1.05, greater than 1.1, greater than 1.15, greater than 1.2, greater than 1.25, greater than 1.3, greater than 1.35, greater than 1.4, greater than 1.45, greater than 1.5, greater than 2.0, greater than 2.5, and greater than 3.0.

30. A method according to any one of claim 29, wherein the AXL binding agent is a soluble AXL variant polypeptide.

31. A method according to claim 30, wherein the soluble AXL variant polypeptide lacks the AXL transmembrane domain; lacks a functional fibronectin (FN) domain; has one or more than one Ig1 domain and, optionally, one or more than one Ig2 domain; and has a set of amino acid modifications of the wild-type AXL sequence (SEQ ID NO:1), selected from the group consisting of:
1) Gly32Ser, Asp87Gly, Val92Ala, and Gly127Arg,
2) Glu26Gly, Val79Met, Val92Ala, and Gly127Glu; and
3) Gly32Ser, Ala72Val, Asp87Gly, Val92Ala, and Gly127Arg;
wherein said modification increases the affinity of the AXL polypeptide binding to Growth arrest-specific protein 6 (GAS6).

32. A method according to claim 31, wherein the soluble AXL variant polypeptide is fused to an Fc region.

33. A method according to claim 29, wherein the AXL binding agent is administered in combination with cytoreductive therapy.

34. A method according to claim 33, wherein the cytoreductive therapy is radiation therapy.

35. A method according to claim 29, wherein the AXL binding agent is administered in combination with a chemotherapeutic agent; wherein the chemotherapeutic agent is selected from the group consisting of: daunorubicin, adriamycin (doxorubicin), epirubicin, idarubicin, anamycin, MEN 10755, etoposide, teniposide, vinblastine, vincristine, vinorelbine (NAVELBINE); vindesine, vindoline, vincamine, mechlorethamine, cyclophosphamide, melphalan (L-sarcolysin), carmustine (BCNU), lomustine (CCNU), semustine (methyl-CCNU), streptozocin, chlorozotocin, cytarabine (CYTOSAR-U), cytosine arabinoside, fluorouracil (5-FU), floxuridine (FUdR), thioguanine (6-thioguanine), mercaptopurine (6-MP), pentostatin, fluorouracil (5-FU), methotrexate, 10-propargyl-5,8-dideazafolate (PDDF, CB3717), 5,8-dideazatetrahydrofolic acid (DDATHF), leucovorin, cisplatin (cis-DDP), carboplatin, oxaliplatin, hydroxyurea, gemcitabine, and N-methylhydrazine.

36. A method according to claim 29, wherein the AXL binding agent is administered in combination with immunotherapy; wherein the immunotherapy is selected from the group consisting of: treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAGS, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

37. A method according to claim 29, wherein the AXL binding agent is administered in combination with a poly(ADP-ribose) polymerase (PARP) inhibitor; wherein the PARP inhibitor is selected from the group consisting of: ABT-767, AZD 2461, BGB-290, BGP 15, CEP 9722, E7016, E7449, fluzoparib, INO1001, JPI 289, MP 124, niraparib, olaparib, ONO2231, rucaparib, SC 101914, talazoparib, veliparib, WW 46, or salts or derivatives thereof. olaparib, rucaparib, niraparib, talazoparib and veliparib.

38. A method according to claim 29, wherein the AXL binding agent is administered in combination with pegylated liposomal doxorubicin (PLD), wherein the combination has a synergistic effect.

39. A method according to claim 29, wherein the AXL binding agent is administered in combination with paclitaxel, wherein the combination has a synergistic effect.

40. A method according to claim 29, wherein the cancer overexpresses the biomarker GAS6 and/or AXL.

41. A method according to claim 29, wherein the cancer is selected from the group consisting of a recurrent cancer, a platinum resistant cancer, and a chemoresistant cancer.

42. A method according to claim 29, wherein the cancer is selected from the group consisting of B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.


SEQUENCE LISTING (2 sequences)

SEQ ID NO: 1 - protein, 887 aa, Homo sapiens
MGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 426 aa, Artificial
  soluble AXL polypeptide-Fc fusion
EESPFVSNPGNITGARGLTGTLRCQLQVQGEPPEVHWLRDGQILELVDSTQTQVPLGEDEQGDWIVASQLRITSLQLSDTGQYQCLVFLGHQTFVSQPGYVRLEGLPYFLEEPEDRTVAANTPFNLSCQAQGPPEPVDLLWLQDAVPLATAPGHGPQRSLHVPGLNKTSSFSCEAHNAKGVTTSRTATITVLPQQGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG